(19) United States
(12) Patent Application Publication
(10) Pub. No.: US 2021/0214702 A1
(43) Pub. Date: Jul. 15, 2021

(54) PROCESS FOR PRODUCTION OF RECOMBINANT TNK-TPA BY PACKED-BED PERFUSION SYSTEM

(71) Applicant: GENNOVA BIOPHARMACEUTICALS LIMITED, Pune, Maharashtra (IN)

(72) Inventors: Kameleshwar Prasad SINGH, Pune, Maharashtra (IN); Krishnakumar SUBBIAH, Pune, Maharashtra (IN); Sanjay SINGH, Pune, Maharashtra (IN); Santosh DESHPANDE, Pune, Maharashtra (IN)

(73) Assignee: GENNOVA BIOPHARMACEUTICALS LIMITED, Pune, Maharashtra (IN)

(21) Appl. No.: 17/058,960

(22) PCT Filed: May 21, 2019
(86) PCT No.: PCT/IN2019/050404

§ 371 Date: Nov. 25, 2020

(30) Foreign Application Priority Data
Jun. 1, 2018 (IN) 201821007692

(51) Int. Cl. C12N 9/50; C12M 1/12; C12M 1/00

(52) U.S. Cl. CPC C12N 9/50; C12Y 304/21068; C12M 29/04; C12M 29/10; C12M 25/18

(57) ABSTRACT

The present invention pertains to an economic packed-bed perfusion system for the production of pharmaceutical grade of recombinant TNK-tPA. The present invention involves a cell culture process utilizing CHO cells more specifically in a micro/macro carriers based packed-bed perfusion system. The process of the present invention results in optimum cell growth and maintenance, and minimal build-up of toxic by-products such as lactate and ammonia. The system of the present invention discloses optimized process parameters to enable a resultant TNK-tPA with high yield and pharmaceutical grade purity. The process of the present invention is industrially applicable and possesses economy of scale.

### FIELD OF THE INVENTION

The present invention relates to culturing of cells using perfusion method. In particular, the present invention relates to a novel process for producing recombinant TNK-tPA by packed-bed perfusion system.

### BACKGROUND OF THE INVENTION

Mammalian cells containing a nucleic acid that encodes a recombinant protein are often used to produce therapeutically or commercially important proteins. Although, several high throughput cell culture systems have been used within the biotechnology industry using fed batch processes, there are a few users of perfusion based cultivation processes. Bioreactors support a biologically active environment conducive for biochemical processes involving biological organisms or biochemically active substances derived from such organisms. The bioreactors are typically operated either as batch/fed-batch or in perfusion mode. In the recent years, perfusion process has become an increasingly accepted cell culture process due to its several advantages in terms of high cell density growth, increased productivity, long term production and suitable cell culture conditions.

Perfusion cultivation of animal cells, in particular, mammalian cells with high density viable cells with less cell aggregation and obtaining an even culture of a suspension of single cells without visible aggregates is a very difficult task and depends upon the balancing of various conditions and components of the bio-reactor system. It is important that the method of the bio-reactor should provide an in vitro, continuous, universal and modular system for production of cultured cells based on a scale free model to respond to the requirements of pharmaceutical and bio-technology industries. The method should be capable of being automatic, possess real time control, capable of being run for extended period of time with little or no human intervention, have adequate production control, require reduced volumes of media and suitable of being controlled precisely in terms of time and performance of each bioreactor as well as the whole production plant.

The criticality of scale up is very important in recombinant proteins and the cell culture process parameters in the bioreactor need to be closely regulated to guarantee high protein quality product. Process parameters such as pH, temperature, dissolved oxygen, agitation, glucose uptake rate, amino acid metabolism and accumulation of toxic by-products such as lactate, ammonia, are critical and affect the final quality of the recombinant protein to a great extent.

Several bio-reactor systems in the past have experimented perfusion culturing but report various disadvantages.

For instance, U.S. Pat. No. 6,544,424 discloses a perfusion process for culturing animal cells but US'424 neither discloses nor suggests extreme cell densities which is desired of this system. Furthermore, US'424 discloses that the perfusion process could decrease the attachment and growth of an obstruction on the membrane surface of the hollow fibres and does not disclose any data pertaining to the quality of cell suspension.

Voisier et al. (Biotechnol. Bioeng. 82 (2003), 751-765) presents several cases of high-density perfusion cultivation of suspended mammalian cells by using cell retention devices. However, none of the reviewed articles states that this system or process provide extremely high viable cell densities combined with the extremely high cell viability.

Modified tissue Plasminogen Activator (“TNK-tPA”) also known as Tenecteplase is a 527 amino acid glycoprotein developed by modification of cDNA. Tenecteplase is a recombinant fibrin-specific plasminogen activator that is derived from native t-PA by modifications at three sites of the protein structure. It binds to the fibrin component of the thrombus (blood clot) and selectively converts thrombus-bound plasminogen to plasmin, which degrades the fibrin matrix of the thrombus. Tenecteplase is used for its activity in Acute Myocardial Infraction (“AMI”), Acute Ischemic Stroke (“AN”), pulmonary embolism and for prevention of clotting when catheters are used. Producing TNK-tPA in a large scale in a bioreactor is a challenging task since the culture parameters in a perfusion system has a huge impact in the quality and the quantity of the protein produced.

There are certain perfusion systems that disclose the production of TNK-tPA. However, they pose the disadvantages of not sustaining high cell density, owing to the intermittent cell loss from the reactor because of the inappropriate use of cell retention device, and as well as not maintaining the appropriate cell culture process parameters that is suitable to the cell growth and desired protein expression.

Hence, there is a need to have an effective system for the production of high quality TNK-tPA with desired yield and purity.

### OBJECT OF THE INVENTION

An object of the invention is to provide an economic packed-bed perfusion system for the production of pharmaceutical grade of recombinant TNK-tPA.

### SUMMARY OF THE INVENTION

The present invention pertains to an economic packed-bed perfusion system for the production of pharmaceutical grade of recombinant TNK-tPA. The present invention involves a cell culture process utilizing CHO cells more specifically in a micro/macro carriers based packed-bed perfusion system. The process of the present invention results in optimum cell growth and maintenance, and minimal build-up of toxic by-products such as lactate and ammonia. The system of the present invention discloses optimized process parameters to enable a resultant TNK-tPA with high yield and pharmaceutical grade purity. The process of the present invention is industrially applicable and possesses economy of scale.

### BRIEF DESCRIPTION OF THE DRAWINGS

FIGS. 1A-D shows graphs of TNK-tPA titer (mgL⁻¹) versus perfusion rate (VVD) for four different bioreactor runs. A and B shows the effect of increased perfusion rate on TNK-tPA titer, whereas C and D show the effect of optimal range of perfusion rate on TNK-tPA production.

FIG. 2A-D shows graphs of perfusion rate (VVD) versus lactate concentration (mgL⁻¹) for four different bioreactor runs. A and B shows the effect of increased perfusion rate on lactate concentration, whereas C and D show the effect of optimal range of perfusion rate on lactate concentration.

FIGS. 3A-D shows graphs of TNK-tPA titer (mgL⁻¹) versus lactate concentration (gL⁻¹) for four different bioreactor runs. A and B shows the effect of increased lactate on TNK-tPA titer, whereas C and D show the effect of optimal range of lactate on TNK-tPA production.

FIG. 4A-D shows graphs of the effect of alkali addition (LD⁻¹) on TNK-tPA titer (mgL⁻¹) for four different bioreactor runs. A and B shows the effect of increased alkali addition on TNK-tPA titer, whereas C and D show the effect of decreased alkali addition on TNK-tPA titer.

FIG. 5A-B shows graphs of the effect of dissolved oxygen (DO, %) level on TNK-tPA titer (mgL⁻¹) for two different bioreactor runs. A and B shows the effect of optimal range of dissolved oxygen on TNK-tPA production.

FIG. 6 shows a graph of the effect of agitation (RPM) on TNK-tPA titer (mgL⁻¹) production.

FIG. 7A-B shows graphs of the relationship between residual glucose (gL⁻¹) and TNK-tPA titer (mgL⁻¹) for two different bioreactor runs. A and B shows the effect of optimal range of residual glucose on TNK-tPA production.

FIG. 8 shows a scatter plot of the effect of temperature shifts (Celsius) on TNK-tPA titer (mgL⁻¹). Vertical solid lines represent the time point where the temperature shifts are introduced to promote increased titer of TNK-tPA.

FIG. 9A-D shows graphs of the relationship between perfusion rate (VVD) and alkali addition (LD⁻¹) for four different bioreactor runs. A and B shows the effect of increased perfusion rate on alkali addition, whereas C and D show the effect of decreased perfusion rate on alkali addition.

FIG. 10 A-B shows graphs of the relationship between cell specific perfusion rate (CSPR, pL⁻¹Ce11⁻¹Day⁻¹) and TNK-tPA titer (mgL⁻¹) for two bioreactor culture conditions. A and B shows the effect of increased and optimal ranges of CSPR on TNK-tPA titer production.

FIG. 11 shows a graph of the effect of viable cell density in terms of capacitance measurement (pFCm⁻¹) on TNK-tPA (mgL⁻¹) production.

FIG. 12 shows a graph of viable cell density for a bioreactor run in terms of capacitance measurement (pFCm⁻¹). Vertical dashed line represents the initiation time point of perfusion of serum-free production medium.

### DETAILED DESCRIPTION OF THE INVENTION

The present invention discloses an economic packed-bed perfusion system for the production of pharmaceutical grade of recombinant TNK-tPA.

According to the present invention, perfusion has its conventional meaning in the art i.e. it means that during cultivation, cells are retained by micro/macro carriers that present inside the bioreactor. These carriers not only assist to provide necessary surface area for efficient cell attachment but also to grow the cells at high cell density. During perfusion, fresh nutrient medium is continuously added to the culture and simultaneously the spent medium containing product of interest is removed, while the cells are remained attached with carriers. At high cell density, non-attachable cells may be present in spent medium. In the event of presence of cells with such spent medium, then a cell retention device containing microcarrier screen filter module, in which there is an outflow of liquid having a lower cell density than prior to separation and in which, there is an inflow of cell culture medium, may be used.

The term “microcarrier screen filter” may include a screen filter composed of polysulfone material. The surface area of the screen filter may be in the range of 0.02 to 0.5 m², preferably 0.024 m² and 0.244 m². The mesh size of the screen filter is chosen such that the size of the pores is in the range of 120 μm to 250 μm, preferably 70 μm.

The perfusion system of the present invention may comprise alternating tangential flow within the filter module. Alternating tangential flow as disclosed herein means that the flow is in the same direction i.e. tangential to the hollow fibre, which flow is going back and forth and that there is another flow in a direction substantially perpendicular to the said filter surface. The alternating tangential flow filtration unit enriches the cell concentration by recycling the suspension cells in the culture medium back to the packed-bed system. The disclosure of Alternating Tangential Flow (“ATF”) filtration unit is disclosed in EP 1720972 is referred herein in entirety. So, using both the technology, carriers based cell retention and ATF based cell retention simultaneously, may provide better high cell density perfusion process than using one technology at one time.

The process of the present invention utilizes cell culture mediums suitable for the growth of mammalian cells. The cell culture medium of the present invention comprises salts, amino acids, vitamins, lipids, buffers, growth factors, trace elements and carbohydrates. Suitable medium of the present invention includes IMDM (Iscove's Modified Dulbecco's Medium) and CHO-S-SFM culture medium as growth and production medium, respectively.

In another embodiment, the present invention discloses a process of packed-bed perfusion system for production of recombinant TNK-tPA.

The present invention is a process for the production of pharmaceutical grade of recombinant TNK-tPA by economic packed-bed perfusion system comprising the steps of:
i. Culturing of mammalian cells;
ii. Designing of bioreactor system;
iii. Optimization of perfusion rate, DO level, agitation speed, temperature and pH;
iv. Maintenance of levels of toxic by-products;
v. Management of cell growth and viability;
vi. Extraction of TNK-tPA from the culture media;

wherein, the process of the present invention maintains a high-cell density growth of greater than 140×10⁶ cellsmL⁻¹

(i) Culturing of Mammalian Cells

The mammalian cells of the present invention may be selected from the group comprising CHO-K1, CHO-DG44 and CHO-DXB11 cell lines, preferably CHO-DG44 cell line. The media of the present invention may be selected from the group comprising IMDM (Iscove's Modified Dulbecco's Medium), CHO-S-SFM medium, Dulbecco's Modified Eagle medium (DMEM), Ex-Cell™ CHO medium, PowerCHO™ medium and Hyclone™ medium, preferably Isocve's Modified Dulbecco's Medium (IMDM) and CHO-S-SFM cell culture medium.

The culture of the present invention may be initiated with seed development process to inoculate in the desired scale of bioreactor. The seed culture for the packed-bed perfusion reactor in the present invention may be prepared by sub-culturing the recombinant TNK-tPA producing cell line from the cell bank at a cell density in the range of 8−12×10⁶ cellsmL⁻¹. It may be sub-cultured in 1×T-175, 2×T-175 and 4×T-175 flasks with IMDM as the growth medium. It may be followed by sub-culturing in 2×850 cm², 2×1700 cm² and 4×1700 cm² roller bottles, and then in 2×10 layer stacks (6200 cm²) and 2×40 layer cell stacks (24000 cm²). The seed culture may be prepared at a cell density in the range of 900−1100×10⁶ cellsL⁻¹ by pooling-down the cells from the cell stacks.

(ii) Designing of Bioreactor System

Two packed-bed perfusion reactor units [New Brunswick Scientific (NBS) and iCELLis] with a geometric volume of 40 L and 70 L were used for the production of recombinant TNK-tPA.

The reactors may be operated at a working volume in the range of 30 L or 55 L with the provision of four gases such as CO₂, Air, Nitrogen and Oxygen at a flow rate of 0.01 VVM to 0.2 VVM, wherein CO₂ gas may be utilized to maintain pH in the media and the other gases Air/Nitrogen/Oxygen may be utilized in a mixed proportionate to maintain the level of dissolved oxygen in the media. Importantly, the pressure inside the bioreactor is maintained from 0.1 mbar to 2 mbar. In addition, the reactor contained a packed-bed basket impeller, where micro/macro carriers such as Fibra-Cel®, Cytodex-1, Cytopore-1, Cytopore-2, polyester microfibers and BioNOC II may be loaded as a packing material, preferably Fibra-Cel® disk and polyester microfibers, for efficient cell attachment purpose to enhance the cell growth at high cell density. Further, the reactor may contain a specially designed inlet and outlet ports, wherein the growth/production medium and alkali may be provided separately through any one of the four inlet ports and removal/harvest of the culture media may be processed through one outlet port.

(iii) Optimization of Perfusion Rate, DO Level, Agitation Speed, Temperature, pH

The desired cell density and cell viability may be maintained according to the process and the parameters, set out in the present invention. The perfusion rate of the media of the present invention may be in the range of 0.3 VVD to 9 VVD, preferably 2.5 VVD.

The DO level of the media of the present invention may be in the range of 20% to 80%, preferably 50% to 70%. The agitation of the media of the present invention may be in the range of 150 rpm to 200 rpm, preferably 170 rpm to 190 rpm. The temperature of the media of the present invention may be in the range of 30° C. to 40° C., preferably 33.5° C. to 35° C., more preferably 35.0° C. to 36.0° C. The pH of the media of the present invention may be in the range of 6 to 8, preferably 7.1 to 7.3. The Osmolality may be in the range of 260 mOsmkg⁻¹ to 330 mOsmkg⁻¹, preferably 280-300 mOsmkg⁻¹.

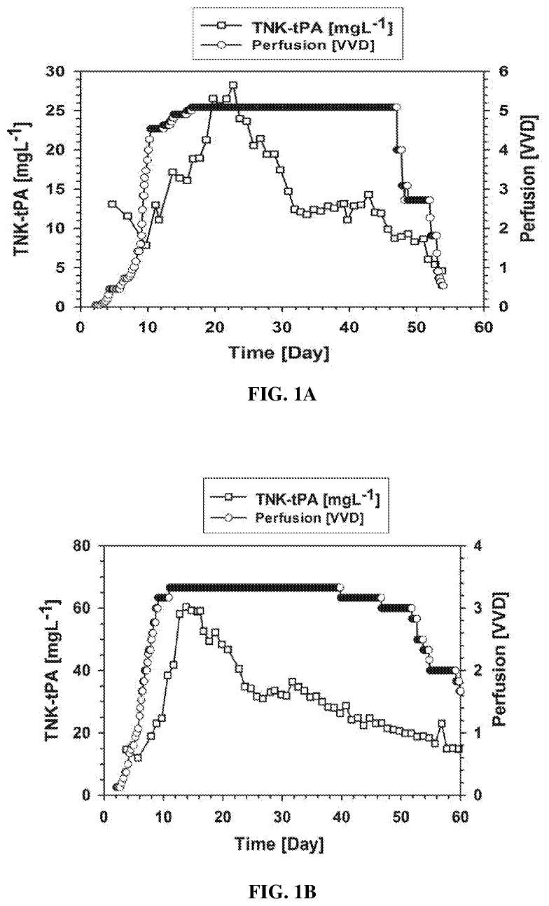

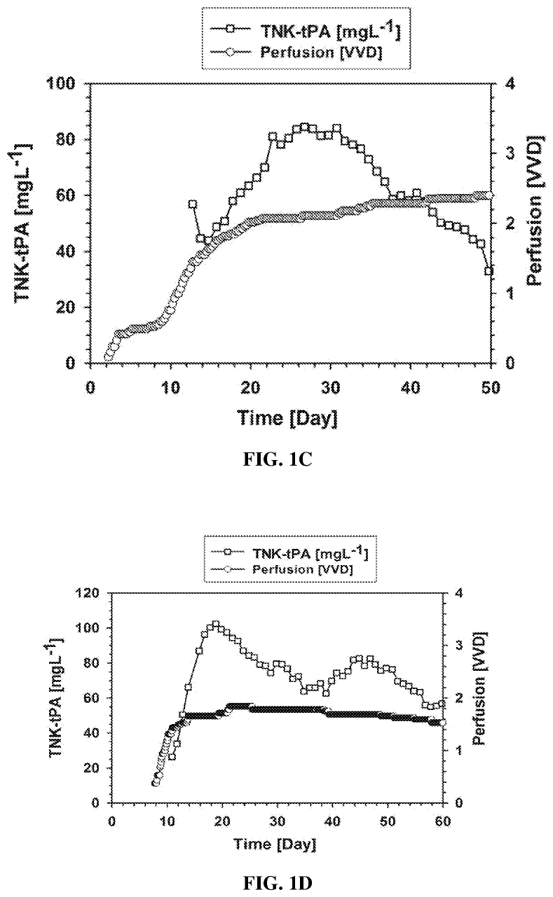

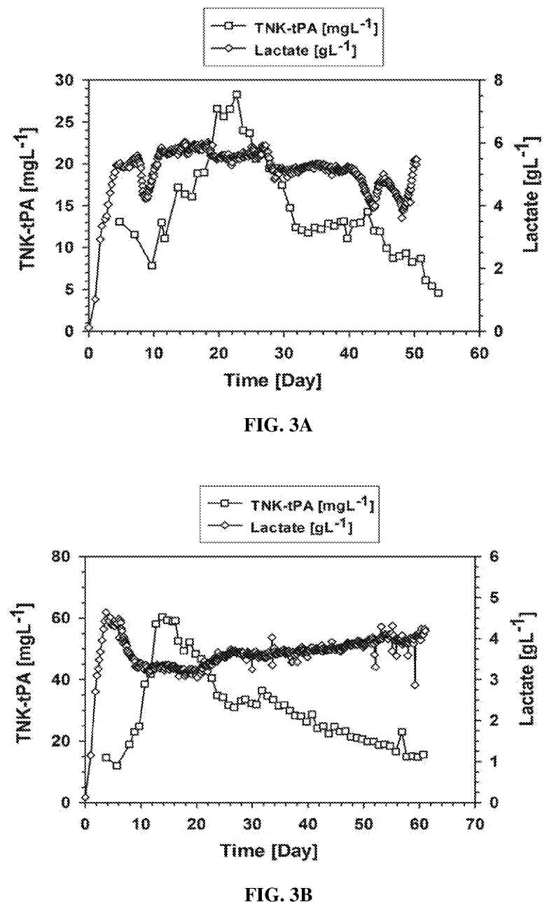

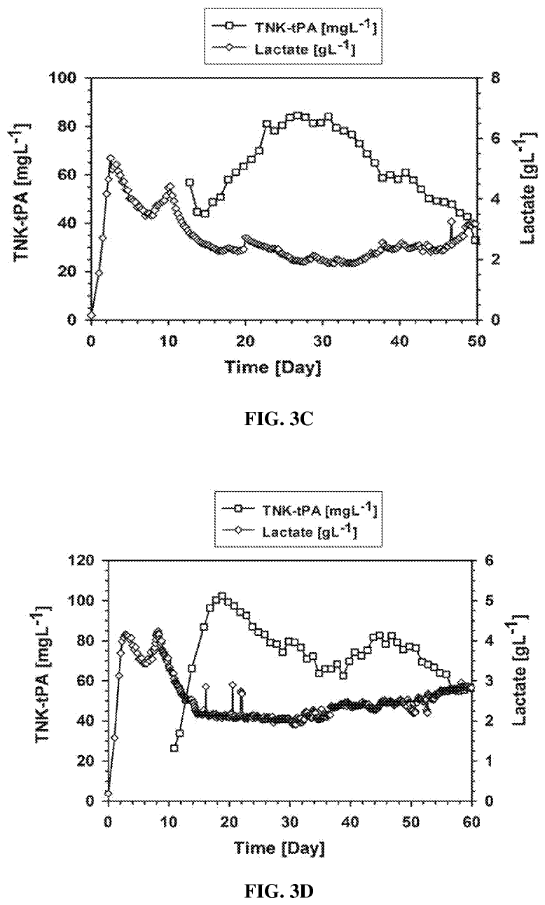

The optimised process of the present invention enables a 52% reduction in perfusion (VVD) of the culture medium for two different bioreactor runs operated under optimal condition, from 5 VVD to 2.4 VVD (FIG. 1A and FIG. 1C) and 3.4 VVD to 1.6 VVD (FIG. 1B and FIG. 1D) respectively. Furthermore, it significantly promotes the TNK-tPA concentration (mgL⁻¹) greater than 2-fold for two different bioreactor runs operated under optimal condition, from 28.28 mgL⁻¹ to 84 mgL⁻¹ and 60.4 mgL⁻¹ to 102.4 mgL⁻¹ respectively. This is evidenced by the results at FIG. 1A-D.

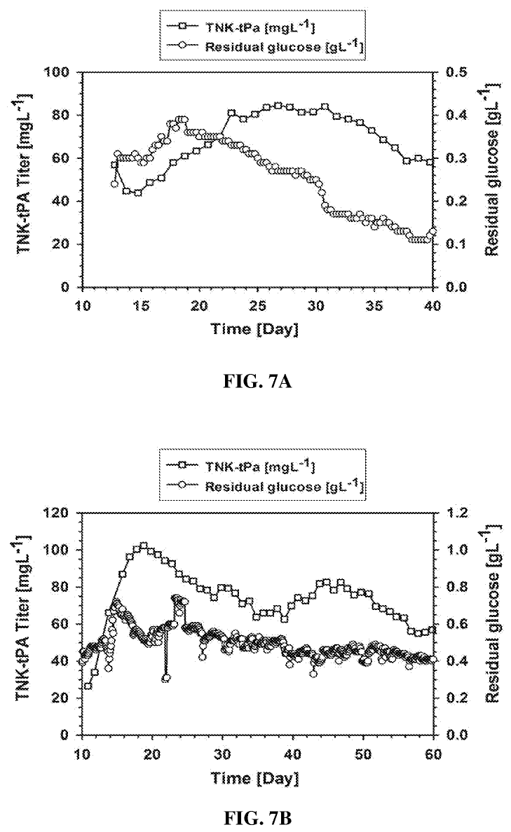

From the prior arts IN01807MU2006A and WO 2012/085933, it discloses that the preferable range of perfusion of production medium is between 2.5 VVD to 5 VVD to maintain the residual glucose in the range of 0.15 gL⁻¹ to 0.75 gL⁻¹ and 0.3 gL⁻¹ to 1.55 gL⁻¹ respectively throughout the production phase. However, the present invention shows an improved optimized process by controlling the perfusion of medium to less than 2.5 VVD based on cell specific perfusion rate feeding strategy, which not only enabled a 52% reduction (FIG. 1A-D) in perfusion of the culture medium, but also led to maintaining of residual glucose in between 0.1 gL^{-l}to 0.5 gL⁻¹(FIG. 7A-B).

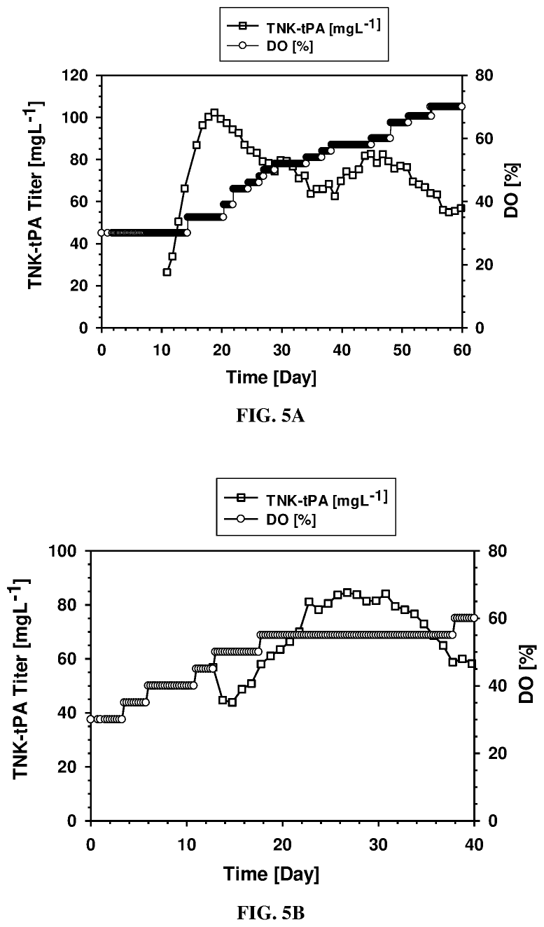

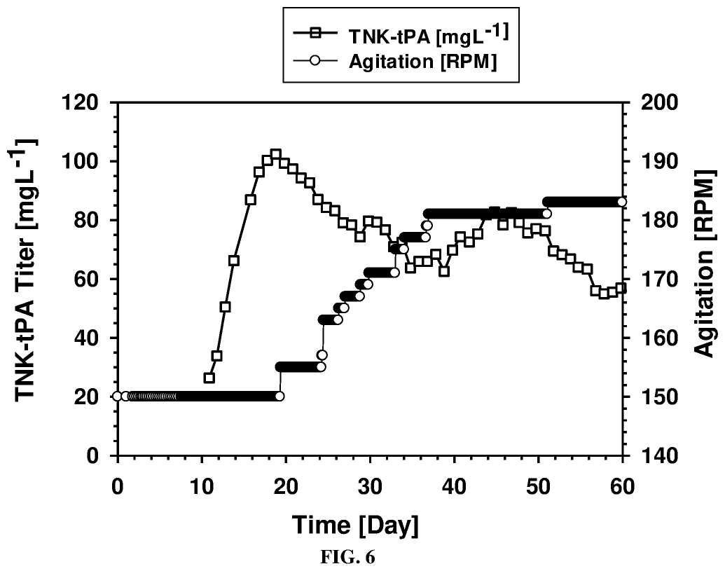

In case of DO and agitation, the prior arts IN 1807/MUM/2006 and WO 2012/085933, discloses that the preferable range is maintained between 10% to 30% and 80 rpm to 120 rpm respectively. However, the present invention maintains DO and agitation at higher preferable range greater than 30% and 120 rpm, which not only assisted in promoting the TNK-tPA productivity greater than 70 mgL⁻¹, but also led to sustaining of TNK-tPA productivity between 60 mgL⁻¹ to 80 mgL⁻¹ for a period of 40 days. This is evidenced by the results at FIG. 5A-B and FIG. 6.

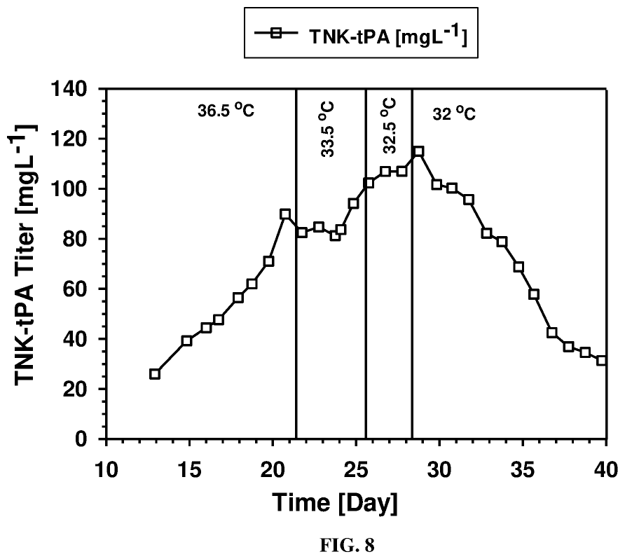

Similarly, from the prior arts IN 1807/MUM/2006 and WO 2012/085933, it claims that the preferable range for temperature is between 31° C. to 39° C. and 33.5° C. respectively, but the present invention preferably maintains the temperature in the lesser range between 33.5° C. to 35° C., which significantly assists in promoting the TNK-tPA productivity greater than 70 mgL⁻¹. In addition, when reducing the temperature below 33.5° C., it significantly affects the TNK-tPA titer, which is evidenced by the results at FIG. 8.

(iv) Maintenance of Levels of Toxic by-products

The present invention advantageously maintains the level of lactate and ammonia in the media. The level of lactate in the media may be maintained less than 3 gL⁻¹, preferably less than 2.5 gL⁻¹. The level of ammonia in the media may be maintained less than 100 mM, preferably 50 mM to 90 mM throughout the lifecycle of the fermentation process, which is in the lifecycle is for a period of 40 to 60 days.

The ratio of lactate:glucose in the media may be in the range of 2:5 to 8:5, preferably 1:5 to 4:5 for a period of 40 to 60 days.

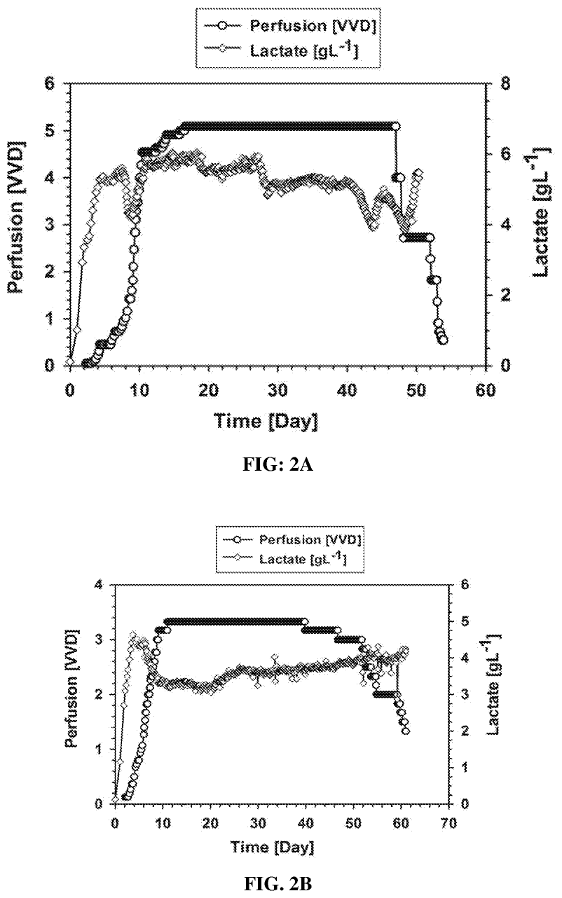

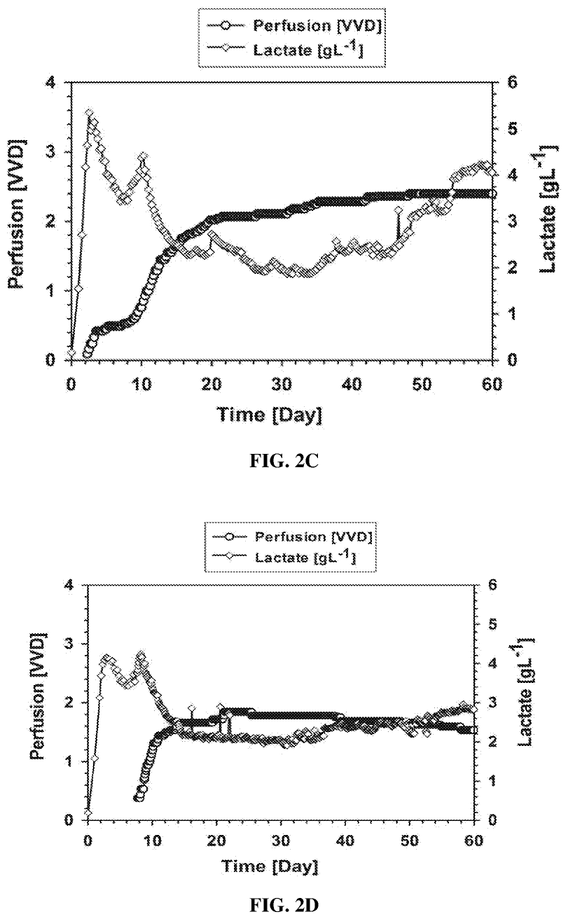

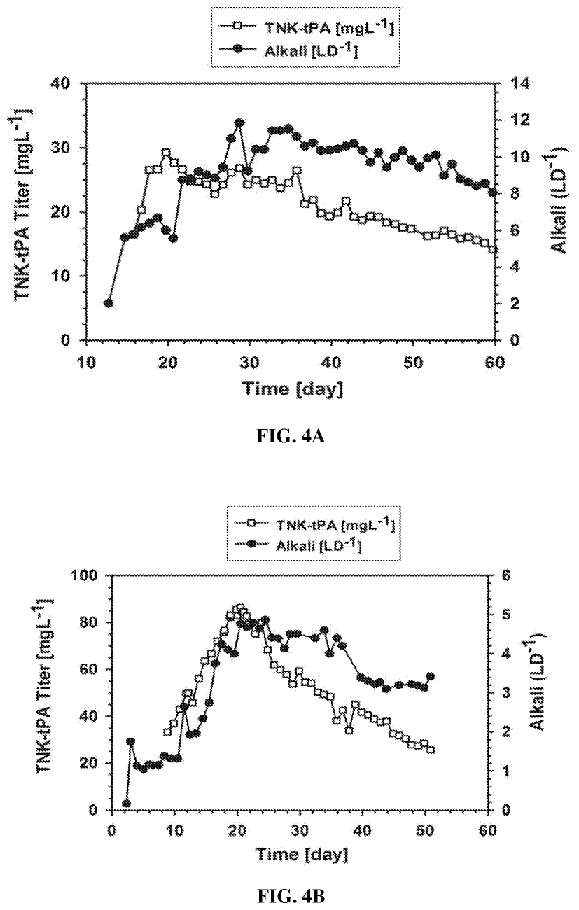

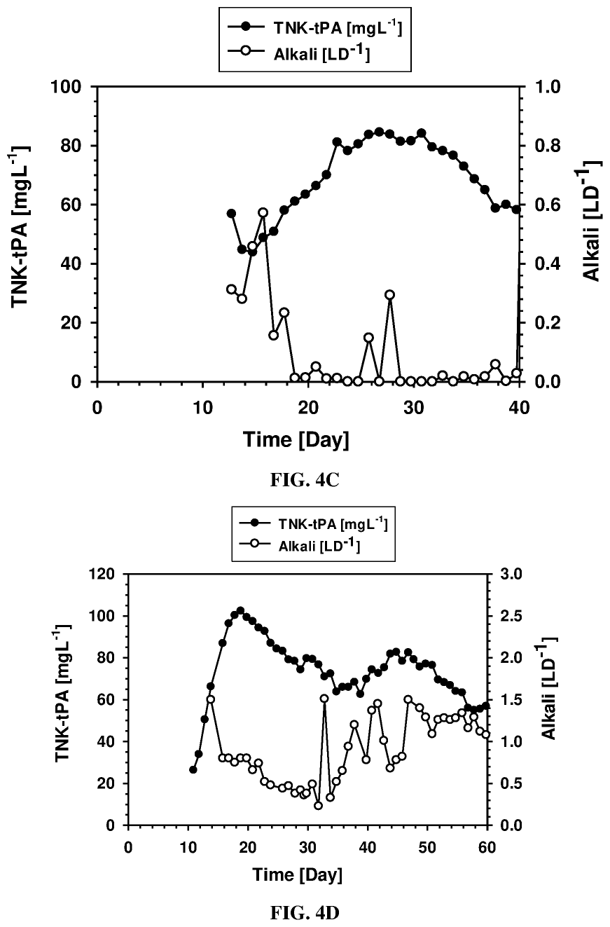

The present invention by maintaining the perfusion to less than 3 VVD significantly reduces the toxic by-product of lactate level (gL⁻¹) by 30% for two different bioreactor runs operated under optimal condition, from 5.9 gL⁻¹ to 4.2 gL⁻¹ (FIG. 2A and FIG. 2C) and 4.2 gL⁻¹ to 2.8 gL⁻¹ (FIG. 2B and FIG. 2D) respectively. With the above same either 30% or 1.4-fold reduction in lactate concentration, it significantly promotes the TNK-tPA concentration (mgL⁻¹) greater than 2-fold for two different bioreactor runs operated under optimal condition, from 28.28 mgL⁻¹ to 84 mgL⁻¹ and 60.4 mgL⁻¹ to 102.4 mgL⁻¹ respectively (FIG. 3A-D).

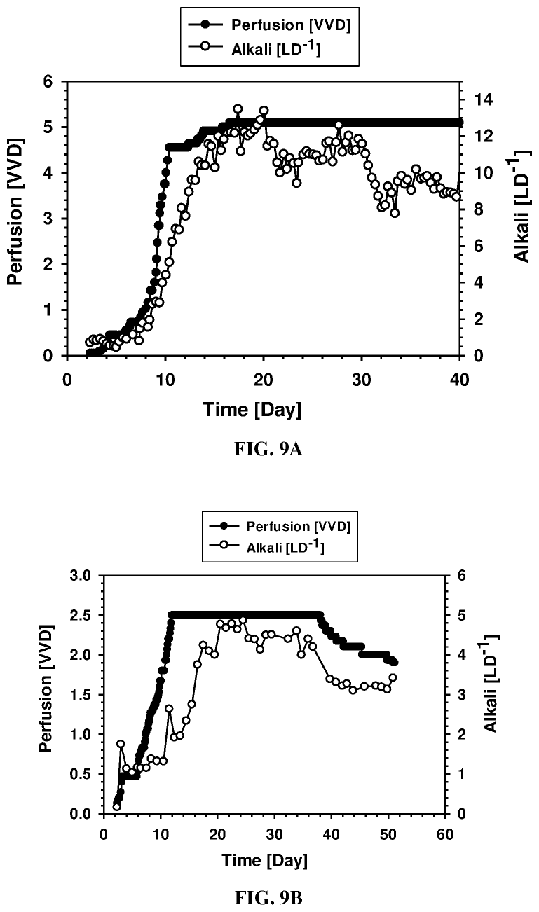

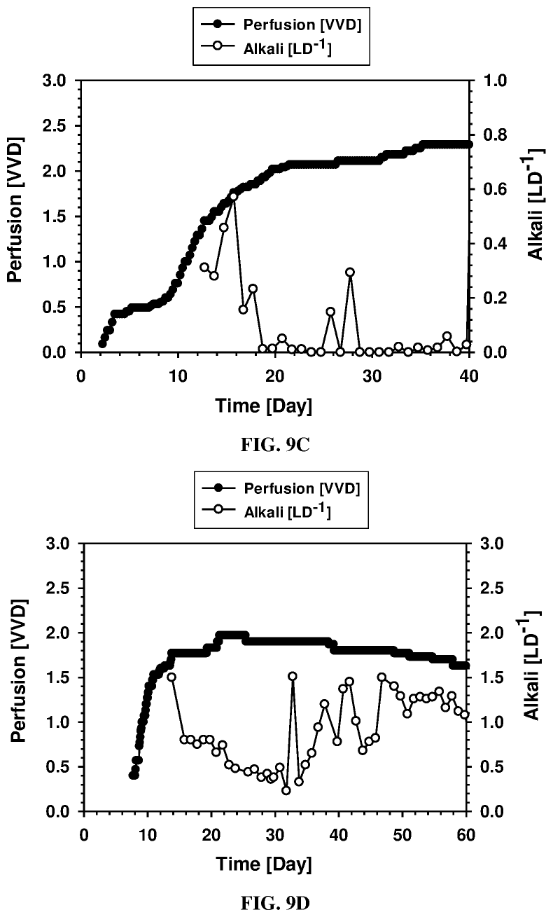

In the present invention, a 1.6 fold reduction in perfusion (VVD) of the culture medium for two different bioreactor runs operated under optimal condition, from 5 VVD to 2.4 VVD and 2.5 VVD to 1.6 VVD respectively, significantly decreases the toxic effect of increased alkali addition by 70% from 12 LD⁻¹ to 3.6 LD⁻¹ and 5 LD⁻¹ to 1.5 LD⁻¹ respectively. This is evidenced by the results at FIG. 9A-D. Furthermore, controlling of alkali addition to less than 3.6 LD⁻¹ significantly promotes the TNK-tPA concentration (mgL⁻¹) greater than 2-fold for two different bioreactor runs operated under optimal condition, from 28.28 mgL⁻¹ to 84 mgL⁻¹ and 60.4 mgL⁻¹ to 102.4 mgL⁻¹ respectively (FIG. 4A-D).

(v) Management of Cell Growth and Viability

The process of the present invention advantageously maintains high-cell density growth of greater than 150 pFcm⁻¹ in terms of capacitance measurement, which is equivalent to a viable cell density of 150×10⁶ cellsmL⁻¹ as per Zhang et al. 2015. The present invention maintains high-cell density growth of above 140 pFcm⁻¹ (in terms of capacitance measurement it is equivalent to 140×10⁶ cellsmL⁻¹ as per Zhang et al. 2015) even with perfusion of serum-free medium throughout the production phase for a period of 40 days (FIG. 12).

In order to achieve high cell density, the value of capacitance in the reactor may be maintained in the range of 50 pFcm⁻¹ to 250 pFcm⁻¹, preferably 170 pFcm⁻¹ to 230 pFcm⁻¹, more preferably 180 pFcm⁻¹ to 200 pFcm⁻¹. The level of residual glucose in the media may be controlled in the range of 0.1 to 2 gL⁻¹, preferably 0.3 gL⁻¹ to 0.4 gL⁻¹, more preferably 0.1 to 0.2 gL⁻¹ through adjusting the perfusion rate from 0.3 VVD to 9 VVD, preferably 3 VVD, more preferably 2.5 VVD.

(vi) Extraction of TNK-tPA from the Culture Media

Extraction of TNK-tPA from the culture media, the harvested culture medium from the packed-bed perfusion reactor may primarily be subjected to two-phase continuous filtration process.

The filtration process may use a polyethersulfone (PES) cartridge filter housing membrane type and the filters may comprise pore sizes of preferably 0.5 μ and 0.2 μ and combinations thereof, and may be stored in a sterile container at 2-8° C. for further use. After filtration, the stored sample may be checked for TNK-tPA content, amount of bacterial endotoxin and bio-burden present, and appearance of sample prior subjecting to purification process.

The stored sample may be subjected to affinity chromatography-I with a column material of preferably Blue Sepharose 6 FF. The affinity chromatogram may be eluted with a buffer that may be selected from the group comprising phosphate buffer, urea and sodium chloride or combination thereof. The pH may be in the range of 7.0-7.6 to obtain partially purified TNK-tPA. The partially purified TNK-tPA may be further subjected other purification processes.

The process of the present invention results in TNK-tPA in terms of specific productivity (calculated based on capacitance measurement) of 1 to 10 pgCells⁻¹Day⁻¹, preferably 3 to 5 pgCells⁻¹Day⁻¹, with a purity of more than 90% using size exclusion chromatography. The present invention results in a 4-fold increase in per cell productivity per day under optimal condition, from 1 pgCells⁻¹Day⁻¹ to 4.2 pgCells⁻¹Day⁻¹, based on perfusion kinetics calculation by considering 1 pFCm⁻¹ of capacitance measurement is equivalent to a viable cell density of 1×10⁶ cellsmL⁻¹ as per Zhang et al. 2015.

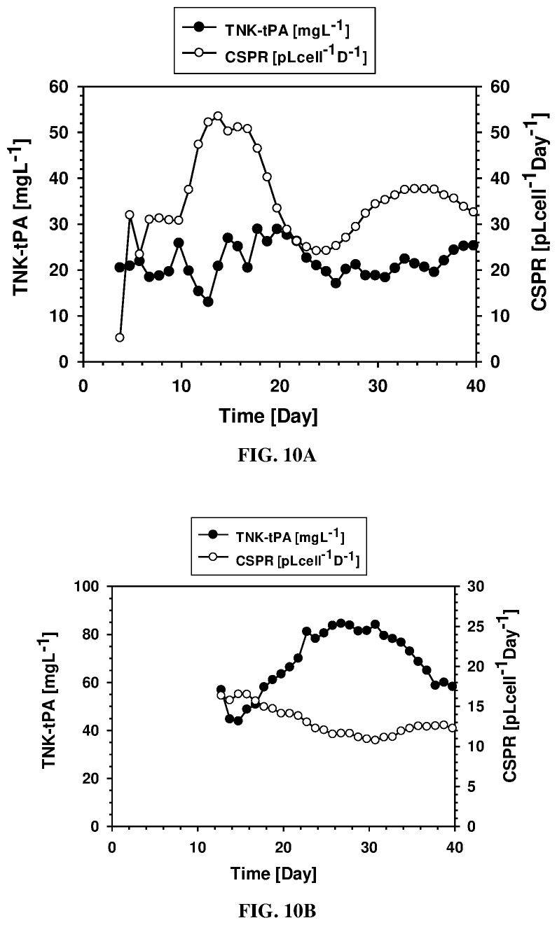

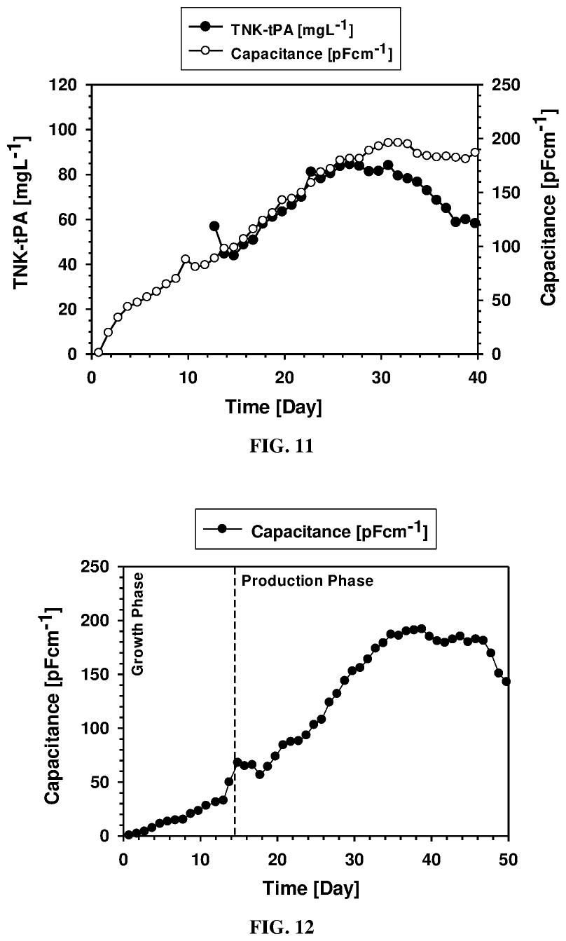

Without being limited by theory, the process of the present invention maintains high cell density with high cell viability, low cell aggregation and low cell death, maintains low levels of toxic by-products and results in TNK-tPA in high yield and high purity. The resultant TNK-tPA is of pharmaceutical grade and suitable for various therapeutic uses indicated for TNK-tPA. The product of the present invention is suitable for use in AMI and AIS. Also, the present invention utilizes a packed-bed perfusion fermentation process without the cell-filtration device provides a highly conducive growth environment for achieving high-cell density growth to a maximum of 190 pFcm⁻¹ (in terms of capacitance measurement it is equivalent to a viable cell density growth of 190×10⁶ cellsmL⁻¹ as per Zhang et al. 2015) without adversely affecting the product concentration (FIG. 11). The process of present invention maintains cell specific perfusion rate (CSPR, pL⁻¹Ce11⁻¹Day⁻¹) of 5 pL⁻¹Ce11⁻¹Day⁻¹to 35 pL⁻¹Ce11⁻¹Day⁻¹, preferably 10 pL⁻¹Ce11⁻¹Day⁻¹to 20 pL⁻¹Ce11⁻¹Day⁻¹ throughout the production phase. Also, a 70% reduction in cell specific perfusion rate (CSPR, pL⁻¹Ce11⁻¹Day⁻¹) from 50 pL⁻¹Ce11⁻¹Day⁻¹ to 15 pL¹Cell⁻¹Day⁻¹, significantly promotes the TNK-tPA concentration greater than 2.5-fold from 28.9 mgL^{-l}to 84 mgL⁻¹ for a bioreactor run operated under optimal condition (FIG. 10A-B).

Advantages of the Present Invention

The present invention provides an economical-feasible approach to produce high-volumetric TNK-tPA productivity combined altogether with less perfusion of production medium and sustained high-cell density growth for longer period.

### EXAMPLES

### Example 1

### Effects of Perfusion Rate, Lactate Accumulation and Alkali Addition on TNK-tPA Productivity

A New Brunswick Scientific (NBS) and iCELLis packed-bed bioreactor with Fibra-Cel® disk and polyester microfibers carriers as solid matrix support was operated at 30L and 55 L working volume respectively. CHO-DG44 cells were grown at high cell density in the range of capacitance 100-250 pFcm¹ throughout the production phase, which is equivalent to a viable cell count of 100-250×10⁶ cellsmL⁻¹ as per Zhang et al. 2015. The perfusion strategy was accomplished through two key aspects, one with perfusion of IMDM medium in the growth phase and other with CHO-S-SFM in the production phase after perfusion of 40-80L IMDM cell culture medium steadily. More importantly, the present invention of the feeding strategy was controlled based on cell specific perfusion rate (CSPR, pL⁻Kell⁻¹Day⁻¹) throughout the production phase for a period of 40 days. However, from the prior art IN01807MU2006A, it discloses that the residual glucose was maintained in between 0.15 gL⁻¹ to 0.75 gL⁻¹ in the entire production phase by appropriately adjusting the perfusion of culture medium, but it does not discloses on any studies related to CSPR based feeding strategy.

In the present invention, feeding of the culture medium at high perfusion rate (FIG. 1A-B) and an optimal range (FIG. 1C-D) were tested. From FIGS. 1A-D, it may be discerned that the perfusion of media at an optimal range less than 3 VVD or a reduction of 52% in perfusion of the culture media for two different bioreactor runs, from 5 VVD to 2.4 VVD (FIG. 1A and FIG. 1C) and 3.4 VVD to 1.6 VVD (FIG. 1B and FIG. 1D) respectively, promotes the TNK-tPA productivity greater than 70 mgL⁻¹ with 2-fold increase in the product concentration, from 28.28 mgL⁻¹ to 84 mgL⁻¹ (FIG. 1A and FIG. 1C) and 60.4 mgL⁻¹ to 102.4 mgL⁻¹ (FIG. 1B and FIG. 1D) respectively. In addition, from FIGS. 2A and 2B, it may be also discerned that increasing of perfusion of media to more than 3 VVD, leads to high amount of lactate accumulation of more than 3 gL⁻¹ (FIG. 2A-B). Moreover, at decreasing rate of perfusion of media to less than 3 VVD, leads to a significant reduction of 30% in lactate accumulation, from 5.9 gL⁻¹ to 4.2 gL⁻¹ (FIG. 2A and FIG. 2C) and 4.2 gL⁻¹ to 2.8 gL⁻¹ (FIG. 2B and FIG. 2D) respectively.

In another study, the effect of critical parameters such as lactate accumulation and alkali addition on TNK-tPA productivity was tested. From FIGS. 3A-D, it may be discerned that controlling of lactate accumulation to less than 3 gL⁻¹ or reducing the lactate level to either 30% or 1.4-fold, from 5.9 gL⁻¹ to 4.2 gL⁻¹ (FIG. 3A and FIG. 3C) and 4.2 gL⁻¹ to 2.8 gL⁻¹ (FIG. 3B and FIG. 3D) respectively, promotes the TNK-tPA titre greater than 70 mgL⁻¹ (FIG. 3C-D), whereas it decreased when the lactate accumulation is more than 3 gL⁻¹ (FIG. 3 A-B). Similarly, from (FIG. 4A-B), it shows that an increase in the addition of alkali to regulate the pH in the media, owing to lactate accumulation, can significantly decrease the TNK-tPA productivity, whereas controlling the alkali addition to less than 3LD⁻¹ (FIG. 4C-D), promotes the TNK-tPA productivity with 2-fold increase in the product concentration for two bioreactor runs, from 28.28 mgL⁻¹ to 84 mgL⁻¹ (FIG. 4A and FIG. 4C) and 60.4 mgL⁻¹ to 102.4 mgL⁻¹ (FIG. 4B and FIG. 4D) respectively. In another study, increasing of perfusion of the media to more than 3 VVD, can significantly increase the volume of alkali addition (FIG. 9A-B), whereas a 1.6 fold reduction in perfusion of the media, from 5 VVD to 2.4 VVD and 2.5 VVD to 1.6 VVD respectively, can lead to a significant reduction of 70% in the alkali addition for two bioreactor runs operated under optimal condition, from 12 LD⁻¹ to 3.6 LD⁻¹ and 5 LD⁻¹ to 1.5 LD⁻¹ (FIG. 9A-D) respectively. Similarly, from (FIG. 10A-B), a 70% reduction in cell specific perfusion rate (CSPR), from 50 pL⁻Kell⁻¹Day⁻¹ to 15 pL⁻Kell⁻¹Day⁻ⁱpromotes the TNK-tPA productivity greater than 70 mg/L⁻¹ with 2.5-fold increase from 28.9 mgL⁻¹ to 84 mgL⁻¹ (FIG. 10A-B), whereas in case of increased CSPR owing to increase in perfusion of the media, leads to significant decrease in the TNK-tPA productivity.

### Example 2

### Effect of Dissolved Oxygen (DO) and Agitation on TNK-tPA Productivity

A similar experimental setup that specified above was used to study the effect of dissolved oxygen (DO) and agitation on TNK-tPA productivity. From FIG. 5 A-B, it may be discerned that maintaining the oxygenation level in between 50 to 70%, promotes the TNK-tPA productivity greater than 70 mgL⁻¹. On the other hand, from FIG. 6, it is shown that controlling of agitation in the range of 150 to 180 rpm, can maintain the TNK-tPA productivity in the range of 60-80 mgL⁻¹ with sustainability of high cell growth and cell viability for a period of 40 to 60 days.

### Example 3

### Controlling of Residual Glucose Level to Increase TNK-tPA Titer

Measuring of residual glucose level in the cell culture process is a critical parameter as it typically signifies the metabolic status of cells, in terms of glucose consumption and energy requirement for cell growth. With the similar experimental setup specified above, the impact of residual glucose level was tested in this study. From FIG. 7 A-B, it may be discerned that controlling of residual glucose in the range of 0.1 to 0.5 gL⁻¹, promotes high cell growth (in terms of capacitance measurement, around 50 to 250 pFcm⁻¹) with a sustained TNK-tPA productivity in the range of 60-80 mgL⁻¹ for a period of 40 to 60 days.

### Example 4

### Optimization of Temperature Reduction for Increased TNK-tPA Production

In this study, the effect of temperature reduction was tested as it is typically considered as a critical factor to enhance the recombinant protein expression. With the similar experimental setup referred above, the culture temperature was initially maintained at 36.5° C. during growth phase of the culture and subsequently reduced up to 32° C. in the production phase. From FIG. 8, it may be discerned that the effective temperature shift from 36.5° C. to 32.5° C. in the production phase, promotes the TNK-tPA productivity greater than 70 mgL⁻¹ and a subsequent drop in the temperature leads to significant decrease in the TNK-tPA productivity (FIG. 8).

### Example 5

### Effect of Cell Growth on TNK-Productivity

In this study, the effect of cell growth on TNK-tPA productivity was tested. As the mode of cultivation type is packed-bed perfusion process, the capacitance measurement in pFcm⁻¹ was considered as a direct measure of the viable cells, such that 1 pFcm⁻¹ is equivalent to 1×10⁶ cellsmL⁻¹ as per Zhang et al. 2015. From FIG. 11, it is discerned that a high-cell density growth to a maximum of 190 pFcm⁻¹ (in terms of capacitance measurement which is equivalent to a viable cell density growth of 140×10⁶ cellsmL⁻¹ as per Zhang et al. 2015), promotes the TNK-tPA productivity greater than 70 mgL⁻¹ (FIG. 11). Similarly, in another study, the perfusion of serum-free medium in the production phase maintains a high-cell density growth of greater than 140 pFcm⁻¹ (in terms of capacitance measurement which is equivalent to a viable cell density growth of 140×10⁶ cellsmL^{-l}as per Zhang et al. 2015) for a period of 50 days (FIG. 12).

1. A process for the production of pharmaceutical grade of recombinant TNK-tPA by economic packed-bed perfusion system comprising the steps of :
i. culturing of mammalian cells;
ii. designing of bioreactor system;
iii. optimization of perfusion rate, DO level, agitation speed, temperature and pH;
iv. maintenance of levels of toxic by-products;
v. management of cell growth and viability;
vi. extraction of TNK-tPA from the culture media;
wherein, the process of the present invention maintains a high-cell density growth of greater than 140×10⁶ cellsmL⁻¹.

2. The process as claimed in claim 1, wherein the mammalian cells are selected from the group comprising CHO-K1, CHO-DG44 and CHO-DXB11 cell lines, preferably CHO-DG44 cell line and the culture medium is selected from the group comprising IMDM (Iscove's Modified Dulbecco's Medium), CHO-S-SFM culture medium, Dulbecco's Modified Eagle medium (DMEM), Ex-Cell™ CHO medium, PowerCHO™ medium and Hyclone™ medium, preferably IMDM and CHO-S-SFM.

3. The process as claim in claim 1, wherein the culture is initiated through seed culture development by culturing the recombinant TNK-tPA producing cell line from the cell bank at a cell density in the range of 8-12×10⁶ cell mL⁻¹, further sub-culturing to 2×850 cm², 2×1700 cm² and 4×1700 cm², further sub culturing to a cell density in the range of 900-1100×10⁶ cellsL⁻¹ by pooling.

4. The process as claimed in claim 1, wherein the bioreactor comprises a working volume of 30 L to 55 Capacity, comprises a mixture of gases selected from air, oxygen, carbon-di-oxide, nitrogen or mixtures thereof, at a flow rate of, 0.01 VVM to 0.2 VVM, and the pressure inside the bioreactor is from 0.1 mbar to 2 mbar.

5. The process as claimed in claim 1, wherein the bioreactor comprises a packed-bed basket impeller, comprising micro/macro carriers selected from the group comprising Fibra-Cel®, Cytodex-1, Cytopore-1, Cytopore-2, polyester microfibers BioNOC II or combinations thereof, preferably Fibra-Cel disk and polyester microfibers, or combinations thereof as packing material.

6. The process as claimed in claim 1, wherein the perfusion rate of the media is in the range of 0.3 VVD to 9 VVD, preferably 2.5 VVD, DO level of the media is in the range of 20% to 80%, preferably 50% to 70%, the agitation of the media is in the range of 150 rpm to 200 rpm, preferably 170 rpm to 190 rpm; the temperature of the media is in the range of 30° C. to 40° C., preferably 33.5° C. to 35° C., more preferably 35.0° C. to 36.0° C.; the pH of the media of the is in the range of 6 to 8, preferably 7.1 to 7.3 and Osmolality is in the range of 260 mOsmkg⁻¹ to 330 mOsmkg⁻¹, preferably 280-300 mOsmkg⁻¹.

7. The process as claimed in claim 1, wherein the level of lactate less than 3 gL⁻¹, preferably 2.5 gL⁻¹, the level of ammonia is less than 100 mM and preferably 50 mM to 90 mM; throughout the entire process for a period ranging from 40 to 60 days.

8. The process as claimed in claim 1, wherein the ratio of lactate:glucose in the media in the range of 2:5 to 8:5, preferably 1:5 to 4:5 for a period of 40 to 60 days.

9. The process as claimed in claim 1, wherein the capacitance is in the range of 50 pFcm⁻¹ to 250 pFcm⁻¹, preferably 170 pFcm⁻¹ to 230 pFcm⁻¹, more preferably 180 pFcm⁻¹ to 200 pFcm⁻¹, perfusion is in the range of 0.3 to 9 VVD, preferable 3 VVD, more preferably 2.5 VVD and residual glucose level in the range of 0.2 gL⁻¹ to 2 gL⁻¹, preferable 0.3 gL⁻¹ to 0.4 gL⁻¹, more preferably 0.1 gL⁻¹ to 0.2 gL⁻¹.

10. The process as claimed in claim 1, wherein the extraction of TNK-tPA from the culture media, is through two-phase continuous filtration process, wherein polyethersulfone (PES) cartridge filter housing membrane type filters are selected with pore sizes of preferably 0.5 n and 0.2 n and combinations thereof.

11. The process as claimed in claim 1, wherein, the TNK-tPA produced is in the specific productivity of 1 to 5 pgCells⁻¹Day⁻¹, with a purity of more than 90% using size exclusion chromatography.

12. The process as claimed in claim 1, wherein the cell specific perfusion rate (CSPR, pL⁻¹Cell⁻¹Day⁻¹) is in the range of 5 pL¹Ce11⁻¹Day⁻¹ to 35 pL⁻¹Ce11⁻¹Day⁻¹ , preferably 10 pL⁻¹Ce11⁻¹Day⁻¹ to 20 pL⁻¹Ce11⁻¹Day⁻¹ throughout the entire production process for a period of 40 day to 60 days.
